(19) United States
(12) Patent Application Publication
(10) Pub. No.: US 2026/0077205 A1
(43) Pub. Date: Mar. 19, 2026

(54) MINIATURIZED MAGNETOELECTRIC-BASED WIRELESS BIOIMPLANT

(71) Applicant: William Marsh Rice University, Houston, TX (US)

(72) Inventors: Jacob T. ROBINSON, Houston, TX (US); Fatima Tayseer ALRASHDAN, Houston, TX (US); Joshua E. WOODS, Houston, TX (US)

(73) Assignee: William Marsh Rice University, Houston, TX (US)

(21) Appl. No.: 19/318,270

(22) Filed: Sep. 3, 2025

Related U.S. Application Data
(60) Provisional application No. 63/694,493, filed on Sep. 13, 2024.

(51) Int. Cl. A61N 1/378; A61N 1/05; A61N 1/08; A61N 1/372

(52) U.S. Cl. CPC A61N 1/3787; A61N 1/05; A61N 1/08; A61N 1/37223

(57) ABSTRACT

The present disclosure relates to a magnetoelectric-based wireless bioimplant (MEWB) that can transmit biological activities sensed via a pair of electrodes to an external transceiver by leveraging magnetoelectric (ME) transducer that is coupled to the implant. The MEWB is also capable of wirelessly receiving power and data via the ME transducer from external transceiver when brought closer to the implanted site (e.g., at few centimeters), thus structuring a batteryless and a leadless bioimplant. MEWB may be implanted epidermally through a pair of electrodes on an organ to capture accurate biological activities. The external transceiver may include a transmitter coil for generating an alternating magnetic field and to transmit downlink data and a receiver coil to sense a backscattered field generated by MEWB and to demodulate uplink data. The overall structure of the disclosed implant is adopted enabling compactness, miniaturization (e.g., <3 cm in length and <4 mm in diameter) and endovascular delivery.

### CROSS-REFERENCE TO RELATED APPLICATIONS

This application claims priority to U.S. Provisional Patent Application Ser. No. 63/694,493 filed Sep. 13, 2024, the entire contents of which are incorporated herein by reference.

### STATEMENT REGARDING FEDERAL FUNDING

This invention was made with government support under Grant No. R01HL144683 awarded by the National Institutes of Health, Grant No. No. FA8650-21-2-7119 awarded by the Defense Advanced Research Projects Agency and Grant No. 2236422 awarded by the National Science Foundation. The government has certain rights in the invention.

### BACKGROUND

Bioelectronics has contributed to various advancements in medical technology, particularly in the development of medical implants that may provide targeted and effective therapies surpassing the capabilities of traditional pharmaceuticals. These devices may be designed to interface with biological tissues, ranging from pacemakers that regulate slow heart rates to neural stimulators delivering electrical impulses and drug delivery systems e.g., insulin pumps for diabetic patients, may offer precise and personalized treatments for various medical conditions. Leveraging these devices may reduce recovery time due to lesser invasiveness as compared to surgical treatments, frequent visits to a medical facility and enhance overall quality of life. However, these devices may pose significant challenges, particularly in terms of miniature device size for decreasing invasiveness, improving accessibility to hard-to-reach body areas and for accommodating constituting components within a limited space without compromising functionality. Achieving the right balance between size, performance, and reliability may be a concern for designing these advanced medical devices.

Additionally, power management and biocompatibility may also contribute to design complexity of the implants. The choice of materials and specific conditions within the body may further complicate the design process. Medical implants often rely on on-board batteries to function, which may limit the extent to which devices can be miniaturized. Due to miniaturized design requirements to be accommodated into various body organs such as endovascular applications, battery capacity may be limited. As a result, batteries may have limited energy storage, leading to frequent recharging or replacement. These procedures can be inconvenient and invasive for patients, disrupting daily life and requiring medical intervention. Moreover, the inclusion of batteries may not only affect the size of the device but also introduce risks such as infections, mechanical failures, and the necessity for periodic replacement surgeries due to movement or breakage. Additionally, the electrical components used for power management may generate heat within the implant, potentially leading to thermal issues that could affect both the performance of the implant and biocompatibility with surrounding tissues.

### SUMMARY

Certain aspects and features of the present disclosure relate to a batteryless, miniaturized and magnetoelectric-based wireless bioimplant (MEWB) that may receive power and data from an external transceiver and transmit sensed biological activities to the transceiver. The apparatus of MEWB may comprise a magnetoelectric (ME) transducer coupled to an electric circuit through a first pair of electrodes that are attached across the ME transducer. The disclosed implant may be mounted on or in the living body epidermally via a pair of second electrodes, hereinafter as sensing electrodes, or endovascularly with a catheter in an organ. The electric circuit may be configured to avail power from the ME transducer, transmit sensing data collected via a second pair of electrodes, also referred hereinafter as sensing electrodes, to an external transceiver and receive data from the external transceiver. The disclosed implant, MEWB, may further include a bias magnet configured to enhance a mechanical strain that in turn may increase the electrical output of the transducer. The bias magnet may be positioned (e.g., parallel, perpendicular or at an angle) with reference to the plane of the ME transducer to enhance the mechanical strain and bias a resonance mode of the ME transducer.

The external transceiver may include a transmitter coil configured to generate a magnetic field, a receiver coil to receive magnetic field generated by the ME transducer, and an external controller configured to operate the working of the external transceiver and remotely control some operations of the MEWB via the ME transducer. The external transceiver may also include a low noise amplifier (LNA) with a fixed gain configured to amplify weak electrical signal from the ME transducer and generate a consistent response by measuring the signal strength. The ME transducer may comprise of at least one layer of magnetostrictive material that is magnetized inducing a mechanical strain when the external magnetic field is applied. This magnetostrictive layer may be coupled with at least one layer of piezoelectric material that may generate an electrical output in response to the mechanical strain from the magnetostrictive material. The magnetoelectric transducer may include an electrode of the first pair of electrodes attached to an outer surface of the magnetostrictive material and another electrode of the first pair of electrodes attached to an outer surface of the piezoelectric material. To modulate the ME transducer during an uplink communication, a switching circuit may be configured that may electrically connect and disconnect an electric load based on the sensing data collected via the sensing electrodes.

In some examples, the at least one layer of magnetostrictive material comprises Metglas and the piezoelectrical material comprises Lead Zirconate Titanate (PZT). The disclosed implant may be encapsulated with a biocompatible material such as medical-grade polyurethane epoxy, where the sensing electrodes may be exposed lying outside the encapsulated area.

While designing the transmitter coils for the disclosed implant, the diameter of the transmitter coil may be large enough for generating strong magnetic field for the implants to operate effectively at depths (e.g., cardiac implants embedded at a few centimeters) without causing harmful effects to the body. The inner diameters of the transmitter and receiver coils may be less than 7 cm while the outer diameters may be at least twice the inner diameters. The transmitter coil may be made up of Litz wires comprising at least two layers of flat spiral coils, where each layer may comprise at least (for example) at least: 5 turns, 8 turns, 10 turns, 12 turns, 15 turns, 17 turns, 20 turns, 22 turns or 25 turns. An inner diameter of the turns may be (for example) at least: 1 cm, 2 cm, 4 cm. or 6 cm; where an inner diameter of the turns is less than 12 cm, 10 cm, 8 cm, or 6 cm. An outer diameter of the turns may be (for example) at least 6 cm, 8 cm, 9.5 cm, 12 cm 16 cm or 20 cm. The receiver coil may include a single layer of flat spiral coil comprising at least 64 turns with an inner diameter of 1.5 cm and outer diameter of 8 cm.

To sense a biological activity using the disclosed implant, an alternating magnetic field may be generated by an external transmitter coil that is coupled wirelessly to the magnetoelectric (ME) transducer. The alternating magnetic field may generate an electrical output across the magnetoelectric transducer due to magnetoelectric coupling, which induces a mechanical strain. The electrical output may be collected via a first pair of electrodes mounted across the ME transducer and rectified to generate a power output. The sensor data including the biological activities may be collected via sensing electrodes, mounted on or in the living body. To compensate for the variations in distance between the external transmitter coil and the disclosed implant, strength of a backscattered signals may be measured via the external controller of external transceiver to determine the distance between the transmitter coil and disclosed implant. Based on a strength of the backscattered signal, the external controller may adjust the gain of a variable gain amplifier for receiving a consistent signal from the disclosed implant during an uplink communication regardless of the variations in distance. The electric circuit of the disclosed implant may include a microcontroller that may modulate the magnetic field of the ME transducer (also referred as backscatter signal) in accordance with the sensor data for transmitting the sensor data to external transceiver during uplink communication. The transceiver may receive the modulated magnetic field and extract the biological activities by demodulating the magnetic field.

In some examples, the modulation of the ME transducer may be achieved by changing its capacitive or resistive load that may affect the frequency or amplitude of the magnetic field. A reactive component such as capacitor may be coupled to the ME transducer for modulation of uplink data. By switching the capacitor in and out of the circuit using a switching circuit, the capacitive load on the ME transducer may be modulated thereby encoding the sensed voltage into a magnetic field. In some instances, the strength of the backscattered field may be measured by the external controller at the output of a low noise amplifier with a fixed gain.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product tangibly embodied in a non-transitory machine-readable storage medium, including instructions configured to cause one or more data processors to perform part or all of one or more methods or processes disclosed herein.

In some embodiments, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present disclosure is described in conjunction with the appended figures:

FIG. 1 shows an illustrative example of a disclosed implant, magnetoelectric-based wireless bioimplant (MEWB), embedded inside a human body.

FIG. 2A shows a block diagram illustrating various components of the disclosed implant in accordance with some embodiments of the present disclosure.

FIG. 2B illustrates exemplary circuit diagrams of one or more components from the FIG. 2A.

FIG. 3 shows an example illustration of an equivalent circuit model of a magnetoelectric (ME) transducer when subjected to an external magnetic field, which is used in the disclosed implant.

FIG. 4 illustrates an exemplary model of a flat spiral coil used as a transmitter coil.

FIG. 5 shows an illustrative block diagram of an adaptive voltage control (AVC) implemented for working of the disclosed implant.

FIG. 6 illustrates screenshots from a practical demonstration of the AVC implemented for the disclosed implant in accordance with some embodiments of the present disclosure.

FIG. 7 illustrates screenshots of an in-vivo open-heart surgery demonstration showing the disclosed implant embedded in a living porcine heart to capture electrogram (EGM) activities.

FIG. 8 shows captured EGM from the in-vivo demonstration of the FIG. 7.

FIG. 9 illustrates an exemplary workflow of the disclosed implant.

### DETAILED DESCRIPTION

The present disclosure relates to a miniaturized, magnetoelectric-based wireless bioimplant (MEWB) that can transmit biological activities sensed via a pair of electrodes to an external transceiver by leveraging a magnetoelectric (ME) transducer that is coupled to the implant. The disclosed implant, referred to interchangeably herein as MEWB, may also wirelessly receive power and data from the external transceiver via the ME transducer, thus structuring a batteryless bioimplant. The disclosed implant may receive a consistent power and maintain a stable communication regardless of the distance variations between the external transceiver and the implant by leveraging an adaptive voltage control mechanism. MEWB may be implanted epidermally through a pair of electrodes on an organ to capture accurate biological activities, for example, capturing electrograms (EGM) for sensing cardiac activities and electrogastrogram (EGG) for sensing electrical activities of stomach muscles. The disclosed implant may comprise an ME transducer coupled via a pair of electrodes to an electric circuit, also referred as system-on-chip (SoC), a bias magnet as strain enhancer and a pair of sensing electrodes. The disclosed implant may be encapsulated with a biocompatible material such as medical-grade polyurethane epoxy avoiding adverse damage to surrounding tissues and maintaining the structural integrity of the circuit components within the implant. The sensing electrodes may be kept outside the encapsulated area for capturing biological activities.

The overall structure of the disclosed implant is adopted for enabling compactness and miniaturization (e.g., <3 cm in length and <4 mm in diameter) such that it may be delivered endovascularly and anchored securely to a specific location within the body. The endovascular delivery may reduce recovery time and risk of complications by making it less invasive compared to open surgeries. For example, MEWB may be implanted through the femoral vein and anchored within the heart chamber to receive stimulation from the external transceiver similar to a pacemaker for the heart. Similarly, it may serve as a gastric electric stimulator delivered endoscopically and anchored to the stomach wall for providing stimulations to regulated digestion or to treat conditions such as gastroparesis. Other applications may include delivering MEWB through relevant arteries e.g., renal artery for kidney, pulmonary artery for lungs, urinary tract for bladder and then anchored in place to stimulate nerve activity or monitor and sense relevant biological activities for treating various conditions. For example, treating hypertension for kidneys by disrupting the renal nerves that may contribute to high blood pressure.

Magnetoelectric-based medical implant, such as the one disclosed, embedded on different organs may enable a mechanism to receive continuous and reliable power supply from external sources avoiding invasive procedures for the replacement of batteries. ME films may be configured as a transducer for wirelessly receiving power from the external transceiver and for communication with the external transceiver. For example, MEWB may transmit sensed voltages (e.g., biological parameters, temperature) to the external transceiver during uplink communication and/or deliver programmable electrical stimulus or commands during downlink communication from the external transceiver to the MEWB. The ME transducer may be comprising at least one layer of magnetostrictive material configured to be magnetized inducing a mechanical strain under the influence of the external magnetic field that is generated by the transceiver. On magnetostrictive layer, there may be at least one layer of piezoelectric material configured to generate an electrical signal in response to the mechanical strain from the adjacent layer of magnetostrictive material. This coupling between magnetostrictive and piezoelectric may be leveraged to wirelessly transfer power and data to and from MEWB through alternating magnetic field.

For powering the disclosed implant embedded on or in a living body and receiving the sensed activity, the external transceiver may be brought closer to the implanted site (e.g., at smaller depths of the order of centimeters). The external transceiver may comprise of an ME-based power transmitter (TX) that includes a transmitter coil for generating an alternating magnetic field and to transmit downlink data (i.e., commands or stimuli), an external controller and an ME-based backscatter receiver (RX) that may include a receiver coil to sense the backscattered magnetic field generated by MEWB and to demodulate uplink data (i.e., sensed biological activities). The external controller may synchronize both ME power TX and backscatter RX modules and remotely program the operation of MEWB by controlling the generation of the applied magnetic field.

The magnetostrictive layer of the ME transducer may generate vibrations under the influence of external magnetic field that may alter its magnetization. These changes in magnetization may result in emission of waves that may reflect back to the source (transceiver) thereby creating a backscattered field generated by the ME transducer. This backscattered field can be used for uplink data transfer to the external transceiver. However, during the time when the external magnetic field is on, the ME materials may vibrate at the same frequency as of applied magnetic field. For separating the backscattered signal from the external magnetic field, the backscattered signal may be measured during off-time of the external magnetic field. During this time, the ME transducer may release stored mechanical resonance energy as decaying voltage at its mechanical resonance frequency irrespective of the frequency of the magnetic field. Uplink data transmission may be achieved by modulating the magnetic, electric or mechanical properties of magnetostrictive layer, piezoelectric layer or both. In some examples, the backscatter signal may be modulated by adjusting the capacitive load on the ME transducer that changes the mechanical resonance frequency of the transducer that in turn may change the frequency of the backscattered signal.

In some aspects of the present disclosure, when the external transceiver is brought closer to the disclosed implant, the transmitter coil within the transceiver may generate an alternating magnetic field that induces an electrical response in the ME transducer coupled to the MEWB. This electrical response or an alternating current (AC) voltage may be collected via the pair of electrodes mounted across the ME transducer and interfaced to SoC of the MEWB. SoC may comprise of a power management module that may rectify AC to a DC voltage and may further generate a stable power supply by leveraging a low-dropout (LDO) voltage regulator. The rectified voltage may also be stored to a charge storing element for energy harvesting and stimulating the disclosed implant when used as a stimulator. The sensing electrodes attached to the SoC of MEWB may sense the biological activities or stimulate the underlying tissue or nerve. The backscattered signal generated by the ME transducer in response to the external magnetic field may be received at a low noise amplifier (LNA) of the external transceiver with a fixed gain for amplifying weak signals. The strength of the received backscattered signal is inversely proportional to the distance between the MEWB and the external transceiver. The disclosed implant may receive the downlink data and the power simultaneously. During the time interval of the first downlink bit, the external controller may detect the backscattered signal strength received from the output of the LNA and accordingly adjust the gain of a voltage gain amplifier (VGA) dynamically.

During the uplink communication, when the implant is to transmit sensed biological activity to the external transceiver, a microcontroller included in SoC of the implant may use the sensed voltage to modulate the backscattered signal generated by the ME transducer. The modulation of the ME transducer may be achieved by changing its capacitive load that may affect the frequency or amplitude of the magnetic field. A capacitor may be coupled to the ME transducer for modulation of uplink data. By switching the capacitor in and out of the circuit using a switching circuit, the capacitive load on the ME transducer may be modulated to encode the sensed voltage into a magnetic field (also referred as backscattered field). The external transceiver may pick up this modulated backscattered field by the receiver coil and perform adjustment via the voltage gain amplifier to compensate the distance variations between the external transceiver and the implant. The external transceiver may then demodulate the backscattered field to extract the sensed data.

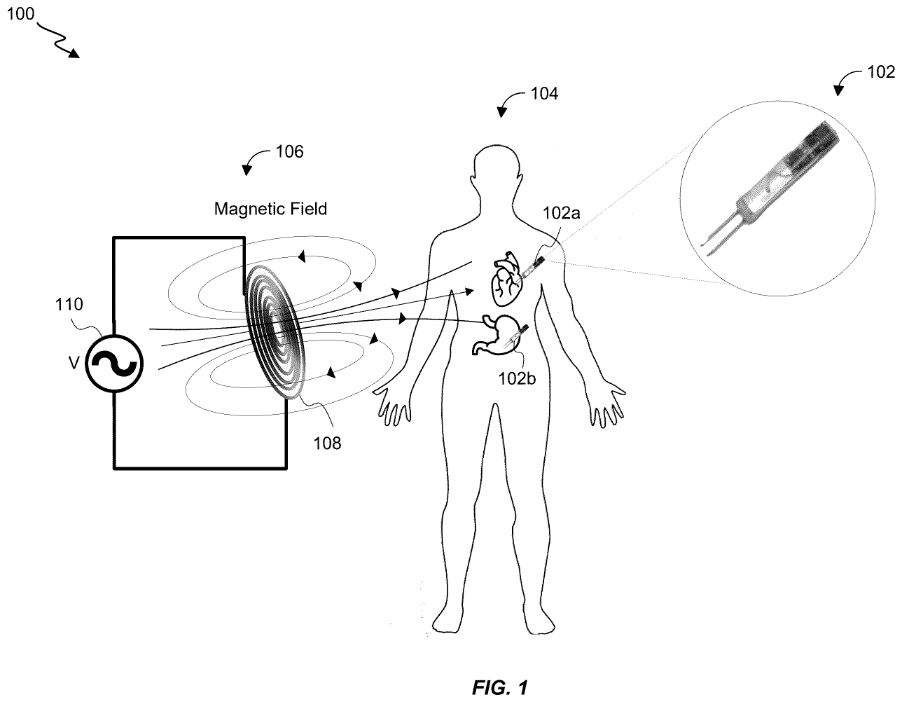

FIG. 1 shows an illustrative example 100 of a magnetoelectric-based wireless implant (MEWB) 102 embedded inside a human body 104. ME materials may be leveraged in energy harvesting from ambient sources such as vibrations, mechanical movements, and magnetic fields that may be utilized for powering low-power electronic devices in remote or inaccessible locations. Magnetoelectric films, which may combine magnetostrictive and piezoelectric properties may offer a solution by efficiently converting high-frequency (e.g., hundreds of kHz) alternating magnetic fields 106 into electrical energy, thereby providing a sustainable power supply for mm-sized devices e.g., pacemakers, insulin pumps, and neural stimulators. ME-based medical implants embedded on different organs for sensing biological parameters may use reliable and continuous power sources avoiding invasive procedures to replace batteries. Wireless power transfer may increase patient comfort by eliminating frequent surgeries, enabling uninterrupted operation of the medical devices. For powering the implants 102a and 102b, an external transmitter coil 108 connected to an alternating voltage source (V) 110 may generate an alternating magnetic field 106. The magnetoelectric film embedded in the implants 102a and 102b, within the vicinity of alternating magnetic field 106, may experience this field as it penetrates through the body 104. The alternating magnetic field 106 may induce a change in magnetization of the magnetostrictive material that may lead to a mechanical deformation or strain in the material.

In response to the mechanical deformation, an electric field and a corresponding voltage may be induced in an adjacent piezoelectric layer. This voltage may be then used (e.g., via electrodes) to power the implants (102a and 102b) eliminating the need for physical connections or battery replacements. ME materials may convert the magnetic energy into electric energy and vice versa, therefore, ME films may be configured as a transducer. For medical applications, ME-based implant may be encapsulated within a hermetic or near-hermetic enclosure. This enclosure may be designed for compatibility with implantation or insertion into the body 104, reducing invasiveness, and miniaturization. ME films may be considered particularly suitable for this application because they can have a high output (e.g., >1 mW) at small sizes (e.g., or the order of cm). However, factors such as the geometry of the magnetoelectric film including height (or ratio of height to width), thickness, orientation relative to incident fields, and closeness of spacing may be adjusted to allow efficient power transfer.

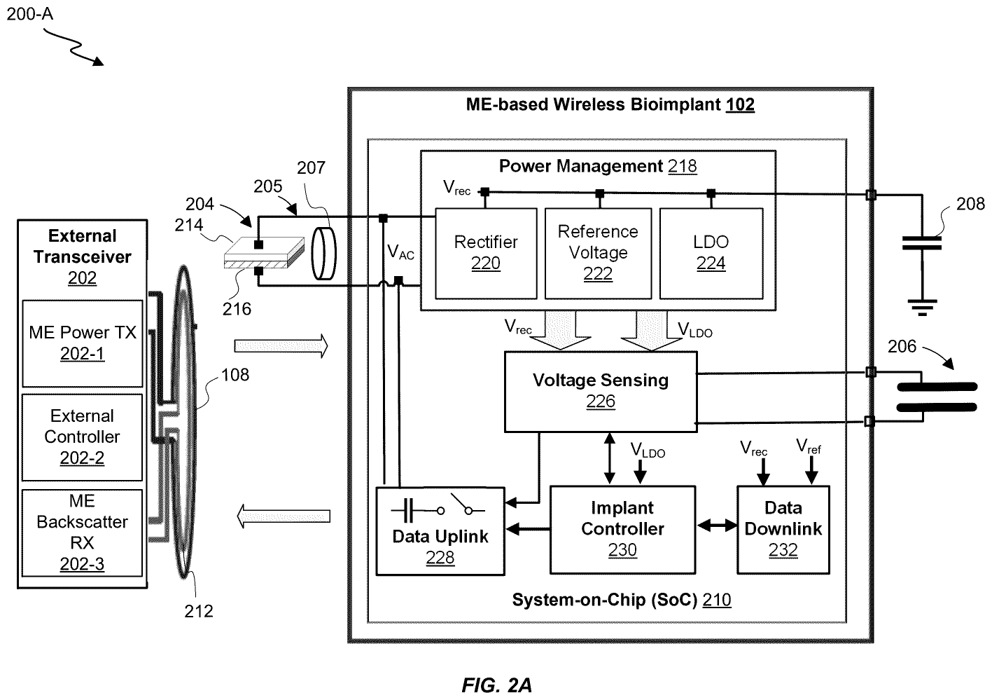

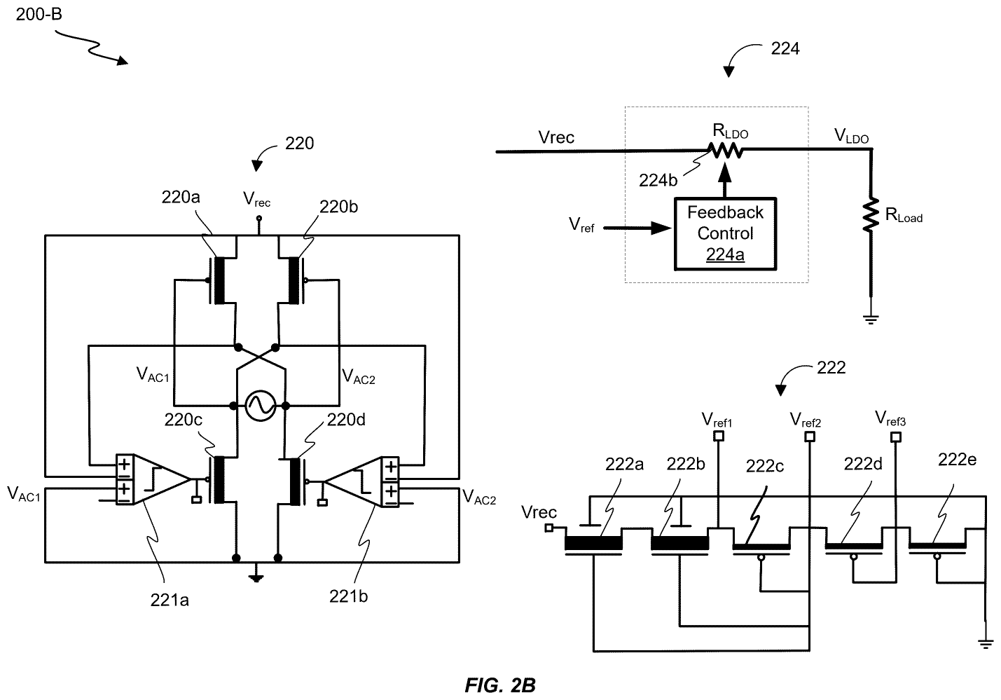

FIG. 2A illustrates an apparatus 200 comprising an external transceiver 202 and an ME-based wireless implant (MEWB) 102 comprising a magnetoelectric (ME) transducer 204, a pair of sensing electrodes 206, a bias magnet 207, a charge storing element (CSE) 208 and a system on chip (SoC) 210. The bias magnet 207 may be configured to enhance a mechanical strain of the ME transducer 204 that in turn may increase the electrical output of the ME transducer 204 that may be collected via a pair of electrodes 205 attached across the ME transducer 204. The placement of bias magnet 207 may be performed in various ways to bias the resonance mode of ME transducer 204, for example, by placing the bias magnet 207 parallel to the plane of the ME transducer 204 can enhance the magnetic field within the plane thereby aligning with the magnetic domains of the material and inducing strain. Alternatively, the bias magnet 207 may be positioned perpendicular to the plane of the ME transducer 204 creating a magnetic field that penetrates the ME material thus affecting the out-of-plane magnetic domains and influencing the mechanical strain differently. The bias magnet 207 may also be positioned at an angle to the plane of the ME transducer 204 to create a complex magnetic field that may interact with multiple magnetic domains, potentially affecting both in-plane and out-of-plane responses.

External transceiver 202 may comprise of an ME-based power transmitter (ME-TX) 202-1, an external controller 202-2 and an ME-based backscatter receiver (ME-RX) 202-3. The ME-TX 202-1 may connect a transmitter coil 108 to apply an alternating magnetic field 106 for power delivery and downlink data. The ME-TX 202-1 may be designed such that it drives a high-frequency (e.g., >10 kHz) biphasic current into the transmitter coil 108 that in turn may generate the alternating magnetic field 106. By maintaining transmitter power levels below a certain power limit e.g., 1 W, operating field strengths such as >1 mT, may be achieved to power the MEWB 102 within the safety limits of the living body 104 at smaller depths (e.g., of the order of centimeters). The ME-RX 202-3 may include a receiver coil 212 to sense the backscattered magnetic field generated by the MEWB 102 and to demodulate uplink data. The external controller 202-2 may synchronize both ME-TX 202-1 and ME-RX 202-3 modules and remotely program the operation of MEWB 102 by controlling the generation of the applied alternating magnetic field 106.

MEWB 102 may transmit sensed voltages (e.g., biological parameters, ambient temperature) to the external transceiver 202 for monitoring during uplink communication and/or deliver programmable electrical stimulus or commands during downlink communication from the external transceiver 202. The ME transducer 204 may be comprising at least one layer of magnetostrictive material (i.e., 214) configured to be magnetized inducing a mechanical strain under the influence of the external alternating magnetic field 106 that is generated by the transmitter coil 112. On magnetostrictive layer 214, there may be at least one layer of piezoelectric material (i.e., 216) configured to generate an electrical signal (VAC) in response to the mechanical strain from the adjacent magnetostrictive layer 214. To wirelessly transfer power and downlink data to MEWB 102 from ME-TX 202-1 through alternating magnetic field 106, coupling between magnetostrictive 214 and piezoelectric 216 may be leveraged.

The vibrations generated in the magnetostrictive layer 214 may result in a change in the material magnetization. This change can be seen as a backscattered field generated by the ME transducer 204, hence leveraging these echoes as backscattered signals can enable uplink data transfer from the MEWB 102 to the external transceiver 202. However, during the on-time of the applied field, the ME materials may vibrate at the same frequency as of applied alternating magnetic field 106. To decouple the generated backscattered field from the magnetic field response, the backscattered field may be measured when the magnetic field is off. Over this period, the ME transducer 204 may dissipate the stored mechanical energy in the form of decaying voltage at its mechanical resonance frequency irrespective of the frequency of the magnetic field. To modulate the backscattered signal for transmitting data during data uplink 228, characteristics of either magnetostrictive layer 214, or piezoelectric layer 216 or both may be modulated. The modulation of the field can be done by controlling the effective mechanical, magnetic, or electric properties of either layer or the composite. In some examples, magnetostrictive layers 214 can be coated with a thin layer of stimuli responsive polymers that respond to external stimuli by shifting the overall mass, thus mechanically modulating the resonance frequency of the MEWB 102.

In other examples, modulating the electric loading conditions may be adopted to tune the resonance frequency of the piezoelectric resonators by changing the electric damping. This is because it may be easier to control this condition inside the body with a simple electronic circuit connected directly to the ME transducer 204. The electric load can be either active load: DC biasing voltage or passive load: inductive, resistive, capacitive, or a combination thereof. In a preferred embodiment, passive loads may be used to maintain the miniaturized footprint of MEWB 102 and avoid any constraints on the power budget. Inductive loads can change both the mechanical and electrical resonance characteristics of the ME transducer 204, however, inductor may cause electromagnetic interference (EMI) problems and is typically bulky, hence, either capacitive or resistive loads may be preferred.

The SoC may include a power management module 218 that is interfaced with the ME transducer 204 configured to manage power and/or harvest energy by charging the CSE 208, which in one exemplary implementation is a capacitor. However, any number of charge storing elements or batteries may be used depending upon the specific application. The power management module 218 may include a rectifier 220 that may be coupled directly to the ME transducer 204 via a pair of electrodes 205, a reference voltage generator 222 connected to the rectifier for generating one or more reference voltages, and an LDO (low-dropout) linear voltage regulator 224 configured to supply a stable and constant voltage (V_{LDO}) despite variations in the input voltage from the rectifier (V_{rec}). The rectifier 220 may first convert the alternating current (AC) voltage (V_{AC}) generated by the ME transducer 204 to a direct current (DC) voltage (V_{rec}) and then store the energy to the charge storing element 208 for high-power stimulation. The reference voltage generator module 222 may take input voltage V_{rec} and generate temperature and supply invariant reference voltages. The LDO 224 may provide a stable supply to subsequent modules reducing power consumption.

For example, the subsequent modules main include voltage sensing module 226, data downlink module 232 and a microcontroller 230. As illustrated in FIG. 2A, voltage sensing module 226 may be coupled with the pair of sensing electrodes 206 that may serve as an interface between MEWB 102 and living body 104 for picking up electrical signals from the body 104 or providing stimulation. For example, for sensing and/or monitoring biological activity, including, but not limited to, heartbeats, temperature, blood flow, motion, and/or any other sensible property of the underlying organ. In some examples, the sensed voltages may be processed by an analog-to-digital converter (ADC) that may leverage a voltage-controlled oscillator (VCO) and then fed to data uplink module 228. The output frequency of VCO may linearly change with the input voltage of the MEWB 102. Alternatively, the sensed voltages may be directly provided to the data uplink module 228 that may be configured to transmit sensed data to the external transceiver 202 through the ME transducer 204.

For transmitting sensed data, the microcontroller 230 may modulate the capacitive or resistive load on the ME transducer 204 to shift the frequency of the backscattered signal. The data uplink module 228 may not include any active-radio components resulting in negligible power consumption (<5 nW). Additionally, one or more transistors such as MOSFETs (Metal-Oxide-Semiconductor Field-Effect Transistors) can be used as switching circuitry to control when and what data is transmitted from the voltage sensing module 226 to the external transceiver 202. The voltage sensing module 226 may be connected to the data uplink module 228 that may be further connected to ME transducer 204 that acts as a transmitter for the uplink data generating a modulated magnetic field (or backscatter signal) in response to the sensing voltage. This modulation may be based on amplitude, frequency, or phase modulation techniques, depending on the design of the ME transducer 204 and the communication needs. The ME-RX 202-3 equipped with a receiver coil 212 may pick up the generated backscatter signal, may perform adjustments based on the distance between the disclosed implant 102 and external transceiver 202, and decode it back into the data format for extracting biological activities, which can then be processed and analyzed.

The MEWB 102 may simultaneously receive power and data. Simultaneous power and data transfers may be constrained by the tradeoff between the efficiency and bandwidth of ME transducer 204. In some examples, these constraints are addressed by modulating the downlink data via a modulation scheme such as time-domain modulation, in which multiple bits are encoded into the single pulse duration to amortize the low switching speed of the ME transducer 204. Additionally, a clock circuit may also be leveraged within the microcontroller 230 that extracts a timing reference from the external transceiver 202 that may not be affected by changes in the process or voltage conditions. This may provide consistent clock signals at the necessary frequencies for the system to function correctly. In some instances, a comparator may be used to compare V_{rec} with one or more reference voltages generating a comparison signal (which is a digital signal) that may be used in data downlink module 232 as a downlink signal. During the downlink signal, the backscattered field may be measured by the external controller 202-2 for measuring the signal strength to estimate the distance between the external transceiver 202 and the MEWB 102.

FIG. 2B illustrates exemplary circuit diagrams of one or more components from the FIG. 2A. The rectifier 220 may convert an AC voltage VAC to a stable direct current (DC) output for the purpose of powering subsequent blocks of SoC 210. The rectifier 220 may comprise of various circuit elements e.g., using one or more diodes as used in half wave or full wave rectification, transformers to step down the AC voltage at a targeted level, capacitors to smooth out pulsating DC output. In an exemplary design of the rectifier 220, AC voltage (VAC) induced on ME transducer 204 that is collected via pair of electrodes 205 is first converted to a DC voltage with an efficient active rectifier driven by 4-input differential comparators 221a and 221b and four transistors 220a, 220b, 220c and 220d. The outputs of the differential amplifiers precisely control and drive the bases of the two transistors (NPN or PNP) 220c and 220d, causing them to conduct or block current in response to the polarity of the AC signal. Typically, a combination of NPN and PNP transistors are used to handle both positive and negative portions of AC signal. The rectified output V_{rec} may be fed to the subsequent blocks for supplying power.

The reference voltage generator (RVG) 222, may provide one or more stable voltage levels for the circuit under consideration. The RVG 222 may be comprised of several components. Different configurations may be possible to design a functional RVG. In the current disclosure, a cascade of five NPN or PNP transistors i.e., 222a, 222b, 222c, 222d, and 222e are incorporated, where V_{rec} is used as input from rectifier circuit 220. This configuration may involve voltage division generating, from a single input voltage V_{rec}, multiple stable reference voltages such as V_{ref1}, V_{ref2}, V_{ref3} that may be a factor of V_{rec}. The output of the RVG circuit 222 may be forwarded to the LDO 224, which may further regulate the V_{rec}. FIG. 2B illustrates an exemplary block diagram of a low dropout (LDO) 224 linear voltage regulator. Low dropout (LDO) linear voltage regulator may be used to provide the stable supply problem for sensors and high-precision analog circuits. It may enable efficient use of energy by regulating an output voltage from a higher voltage source efficiently, thus acquiring a smooth functioning. This may save the sensitive parts from burning out due to fluctuations in the power supply voltage. Referring to FIG. 2B, an exemplary design of LDO is shown comprising a feedback control 224a and a variable resistor R_{LDO} 224b to regulate the input voltage V_{rec}.

The rectifier voltage V_{rec} may be adjusted according to the value of the voltage reference via the feedback control 224a that based on the feedback or a comparison with the reference voltage V_{ref} adjust the value of the output voltage accordingly. This simple feedback control 224a loop may safeguard circuitry from damaging from high voltage and provide safety measures for smooth running of the components of the integrated circuit. LDOs may have multiple functions such as reduction of an incoming supply voltage to the lower voltage for a load R_{Load} (that may represent any subsequent block or circuitry). Another function may be to supply a low-noise voltage source, even in the presence of noise on the incoming power supply or transients in the load.

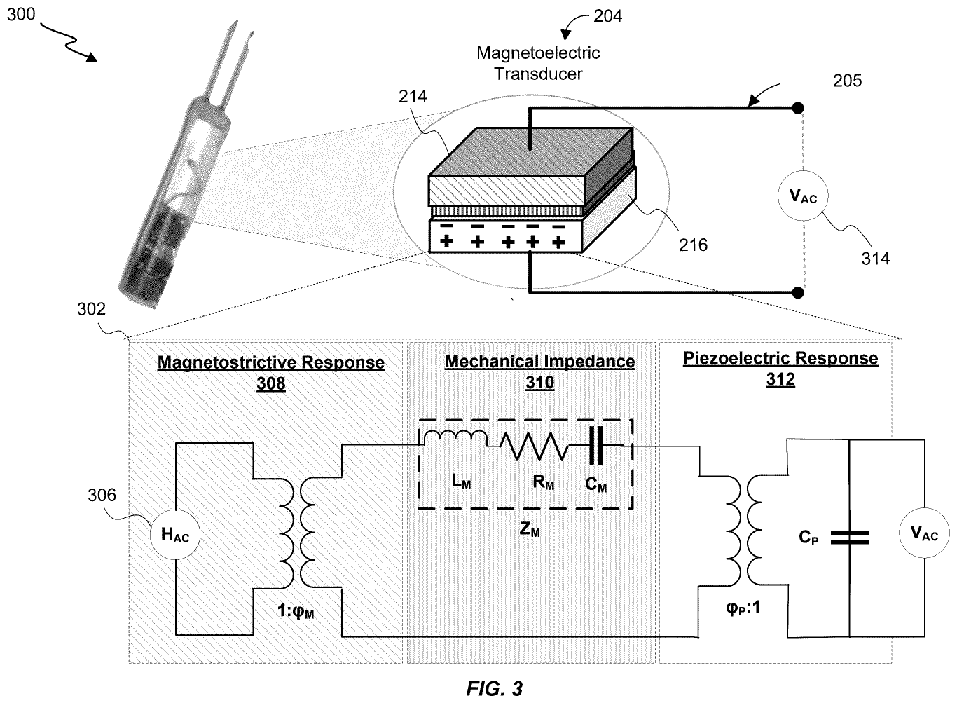

FIG. 3 shows an example illustration 300 of an equivalent circuit model of a magnetoelectric (ME) transducer 204 when subjected to an external alternating magnetic field.

The working principle of ME transducers 204 is the magnetoelectric coupling, where an external alternating magnetic field 106 induces an electrical response and vice versa. The geometry of ME transducers 204 typically includes a layered structure combining one or more layers of magnetostrictive material (i.e., 214) and one or more layers of piezoelectric material (i.e., 216). This composition may generate magnetoelectric effect when an external alternating magnetic field 106 is applied. The magnetostrictive layer 214 is typically made of metallic materials with high permeability such as iron, nickel, boron or their alloys e.g., Metglas, Terfenol-D, which exhibit changes in dimension in response to the applied magnetic field. The magnetostrictive layer 214 may experience changes in strain due to application of alternating magnetic field 106. This strain can induce a mechanical stress within the magnetostrictive layer 214 that may be transferred to an adjacent piezoelectric layer 116 comprising of materials such as lead zirconate titanate (PZT), polyvinylidene fluoride (PVDF), or composites of these material. In response, piezoelectric layer 216 may generate an electric field and a corresponding voltage VAC (e.g., across electrodes 205). The amplitude of the voltage can be modulated by shifting the frequency of the applied alternating magnetic field 106.

Apart from magnetostrictive 214 and piezoelectric 216 layers, ME transducers 204 may include additional layers or components depending upon the specific design and application. These additional layers or components may be incorporated to enhance structural integrity, electrical conductivity or for providing protective coating. For example, a substrate layer that may provide mechanical support, stability, electrical isolation and a foundation upon which the magnetostrictive 214 and piezoelectric 216 layers may be deposited. Moreover, ME transducer 204 may include electrodes 205 that are typically made of conductive materials such as metals (e.g., gold, silver) or conductive polymers. These electrodes may be attached across ME transducers 204 for applying electric signal to the piezoelectric layer 216 and/or extracting electric signals generated by the piezoelectric effect. Additionally, buffer layers that may comprise of thin conductive layers or insulating layers, may be used to optimize the interference between different materials within the ME transducer 204 to reduce stress or prevent diffusion between layers. In some applications, ME transducers 204 may be coated with protective layers or packaged inside an IC (integrated circuit) capsule to enhance durability, resistance to environmental factors (e.g., moisture, corrosion) or biocompatibility for medical applications.

In equivalent circuit model of the ME transducer 204, H_{AC} 306 may represent the amplitude of the applied alternating magnetic field 106 generated by the transmitter coil 108 that is connected to the voltage source V 110. The equivalent magnetic field H_{AC} 306 may induce an elastic excitation or a mechanical stress (used interchangeably herein) in the magnetostrictive layer 214 through a magnetostrictive response 308, which may depend on thickness of the magnetostrictive layer (tm). Losses in the elastic excitation may be represented by an equivalent mechanical impedance Z_{M} 310 (alternating current (AC) equivalence of resistance R_{M}, inductance L_{M} and capacitance C_{M}), which may depend on interface adhesion, mechanical quality factor (Q_{M}), total thickness (t) and thickness ratio (η) of the ME transducer 204. The interface adhesion refers to the bonding strength between two adjacent layers e.g., magnetostrictive 214 and piezoelectric 216 layers. Strong or higher interface adhesion may be consequent in effective transfer of mechanical strain and electrical signals between layers, thereby decreasing energy losses and enhancing power efficiency.

The mechanical quality factor (QM) in ME transducers 204 quantifies the efficiency of mechanical energy storage and dissipation. It is a measure of how well a mechanical system or material stores and releases energy during vibrations or oscillations and may be influenced by factors such as material composition, damping mechanisms (including interface adhesion), and the mechanical design of the magnetoelectric transducer 204. A higher QM may indicate low energy loss and efficient mechanical response, while a lower QM may suggest greater energy dissipation and damping. Similarly, the thickness ratio refers to the relative thicknesses of the layers within the ME transducer 204 structure, such as the magnetostrictive 214 and piezoelectric 216 layers. The thickness ratio may affect mechanical impedance Z_{M} 310 by influencing parameters such as resonant frequencies, mechanical strain distribution, and energy conversion efficiency. Adjusting or fine-tunning the thickness ratio can enhance mechanical resonance and improve the coupling between magnetic and electrical responses, thereby improving energy harvesting or transduction capabilities. The interface coupling factor (denoted as k), directly related to interface adhesion, refers to the degree of coupling and may be influenced by packaging as well, while the mechanical quality factor (QM) may be affected by these factors as well as additional considerations such as clamping. The elastic excitation may be converted into an electric field (E_{AC}) through the piezoelectric response 312, where Cp may represent the capacitance of the piezoelectric layer 216. The piezoelectric response 312 may depend on factors such as thickness of piezoelectric layer (tp). The resulting electric voltage (VAC) 314 can be used to power a device.

Magneto-elastic coupling factor φ_{M}, as illustrated in FIG. 3, quantifies the degree to which the magnetostrictive layer 214 responds to the applied alternating magnetic field 106 by changing its dimension (strain), thus representing the efficiency of the magnetostrictive effect. In other words, the magneto-elastic coupling factor φ_{M} that relates the transfer of the applied alternating magnetic field 106 to an elastic excitation in the magnetostrictive layer 214 depends on the width, magneto-strictive layer thickness, magneto-elastic compliance and piezomagnetic modulus. The induced strain may vary with the frequency and amplitude of alternating magnetic field H_{AC} 306. Magnetoelectric voltage coefficient, defined as the ratio of the change in receiver open-circuit voltage to the change in the applied magnetic field. This magnetoelectric coefficient may be defined in terms of magneto-elastic and electro-elastic coupling factors (i.e., φₘ and φₚ), equivalent mechanical impedance Z_{M} 310 and the load impedance R_{Device}. The typical design of ME transducers contemplates one or several generally rectangular transducers that are positioned vertically in a medical device enclosure (parallel to the incident and generally symmetric magnetic field created by a single coil). One or more techniques are described herein for adjusting different design parameters of the ME transducers to enhance power output in ME transducers 204.

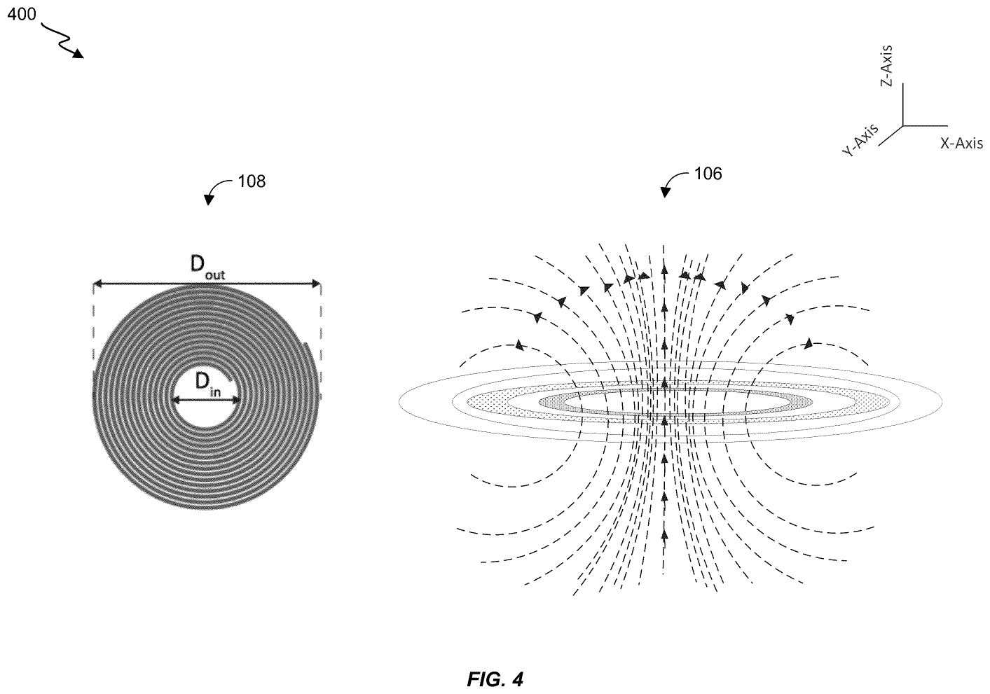

FIG. 4 shows an illustrative example of a transmitter coil 108 along with its magnetic field distribution around the transmitter coil 108. Magnetoelectric-based wireless implant (MEWB) 102 may be used for precise data collection inside the body 104. The transmitter coil 108 may use the ME approach to collect the data wirelessly from a sensing element or monitoring system like MEWB 102. The diameter of transmitter coil 108 may depend on various use cases for a specific application and design. MEWB 102 may be used in medical and scientific applications where they may be inserted on or into the body, for example, MEWB 102 may be used for the heart or stomach. As the heart is at the center and protected by ribs around it so the disclosed implant may be inserted at a deeper level than neural or other sensing elements. While designing the transmitter coil 108, the size and other technical details may be a concern e.g., the diameter may affect the efficiency of power and signal transmission, implying large diameter coils can potentially transmit power and data more effectively over longer distances and may cover more space.

For the disclosed implant 102, the coil diameter may be large enough to generate a magnetic field strong enough that can be sensed at a distance for effective transmission without causing harmful effects to the body 104 and small enough comparable to the overall dimensions of the MEWB 102. Typically, bioimplant designs are sophisticated and may have custom coil diameter to meet the specific application. It may be a flat spiral coil comprising a series of loops or turns that lie in the same plane progressively winding inward or outward, as illustrated in FIG. 4, that can be placed parallel to the implantation site. For the same excitation current the flat spiral coil may have a higher magnetic field at a distance compared to the circular coil that is wounded in cylindrical shape. In addition, the flat spiral coil may have a smaller size and superior misalignment tolerance. The inductance of the flat spiral coil may be calculated using a mathematical function relating to its number of turns, free space permeability, inner and outer diameters.

The MEWB 102 may be sensitive to the direction of alternating magnetic field 106. It may give a strong response when the magnetic field lines are aligned with the ME transducer 204. Using the model of the spiral coil shown in FIG. 4, the spatial distribution of the generated alternating magnetic field 106 may be studied. As shown here, the magnetic field lines may be parallel to the z-direction (when the flat coil is placed along x-y plane) at the center of the coil and may bend toward the coil edges as the radial distance from the center increases. The alternating magnetic field 106 is strong at the center illustrated by closer the field lines and decreases with an increase in the radial distance from the center of the coil. The field distribution may form a symmetric pattern around the coil. As can be seen, the z-component may have a strong value at the center of the coil and. In an example implementation design as shown in FIG. 4, Din and Dout represent an inner and an outer diameter of the coil respectively. A 3D model may be built on a simulation tool for simulating various real time processes and applications for designing the transmitter coil 108 for the disclosed implant. The inner diameter of the transmitter and receiver coils may be comparable to the length of the disclosed implant while the outer diameters may be a multiple of the length of the disclosed implant. In an exemplary design, the transmitter coil 108 may be made with Litz wires and may have two layers. Each layer may have a total of 20 turns with inner diameter may be 4 cm, while the outer diameter may be 9.5 cm. The receiver coil 212 may be a single-layer PCB (printed circuit board) that may have 64 turns. The inner diameter of the coil may be 1.5 cm, while the outer diameter may be 8 cm so it can communicate with the MEWB 102 device effectively.

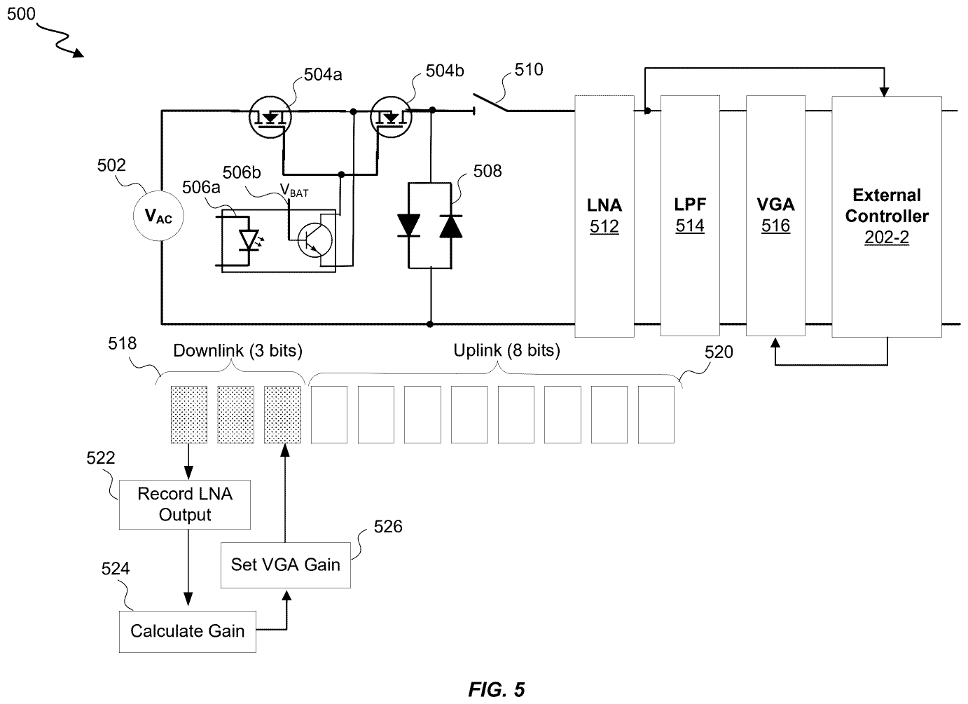

FIG. 5 shows an illustrative block diagram of an adaptive voltage control (AVC) mechanism 500 for the disclosed implant, ME-based wireless bioimplant (MEWB) 102. When external transmitter coil 108, coupled with a magnetoelectric (ME) transducer 204, is used to wirelessly power the MEWB 102, the distance between the transmitter coil 108 and the MEWB 102 may vary due to the movement or changes in the position of MEWB 102 or transmitter coil 108 or both. This variability in distance can affect the backscattered signal strength received by the external transceiver 202 during uplink communication or the received signal strength during downlink communication. By adjusting the gain of a voltage gain amplifier 516, external transceiver 202 may receive a consistent backscattered signal regardless of changes in distance, thus preventing under or over voltage conditions. Adaptive voltage control 500 can dynamically adjust its amplification gain based on the distance between the external transceiver 202 and the MEWB 102.

The circuit in FIG. 5 illustrates an exemplary front-end circuit of the external transceiver 202 performing adaptive voltage control mechanism 500 where an alternating voltage VAC 502 may be induced across receiver coil 212 in response to the received backscattered field generated by the ME transducer 204. The subsequent circuit elements that may include one or more transistors e.g., 502a and 502b with one or more additional diodes 504 that may be forward and reverse biased to rectify positive and negative cycles of the alternating voltage (VAC) (or current) to convert AC to DC. A voltage regular VBAT 506b e.g., a battery or a capacitor may be used to store and stabilize the rectified DC voltage. To visually indicate the voltage regulation or status of the power transfer or as a voltage reference, a light emitting diode (LED) 506a may be included. This configuration of circuit elements as illustrated in FIG. 5 is another embodiment of rectification and voltage regulation similar to the one performed in power management module 218 illustrated in reference to FIG. 2.

When the circuit is closed at 510, the backscattered signal (i.e., voltage or current) may be fed to a linear noise amplifier (LNA) 512 with a fixed gain and a low pass filter (LPF) 514. The LNA 512 may be configured to amplify weak signals (e.g., current or voltage) and generate consistent response, while LPF 514 may filter out high-frequency noise from the signal. The amplified output from LNA 512 or LPF 514 may be supplied to the external controller 202-2, a small processing unit, configured to manage operations of the external transceiver 202 and to dynamically control gain of a voltage gain amplifier (VGA) 516 based on the backscattered signal strength. The adjustment in gain may be larger resulting in increasing the strength of the signal when the distance between the external transceiver 202 and MEWB 102 is larger and similarly, the gain may be smaller at closer distances. In wireless power transfer, the number of bits allocated for downlink and uplink communication may depend on the type of communication and amount of information being transmitted or received. For MEWB 102, downlink communication 518 typically includes control signals, commands and/or a stimulus from the external transceiver 202, less bits may be allocated as compared to uplink communication 520 that may include sending sensor readings to the external transceiver 202. For example, three bits are allocated to downlink communication 518 while eight bits are allocated to uplink communication 520 in an exemplary illustration of FIG. 5.

During the initial downlink bit (i.e., when first bit is sent from the external transmitter coil 108 to the MEWB 102), the received backscattered signal may be amplified by LNA 512 with a fixed gain. Fixing the gain of LNA 512 may be appropriate as signal is amplified within the linear operation range for initial processing, thus avoiding the risk of saturation for subsequent blocks. Moreover, due to fixed gain, the output of the LNA 512 may be reliably attributed to variations in received signal strength indicating how strong the signal is after it has traveled from the MEWB 102. The external controller 202-2 may record the output of the LNA 512, at block 522. Based on the signal strength, the external controller 202-2 may calculate the appropriate gain value for the VGA 516, at block 524, so that the backscattered signal is amplified correctly without causing saturation that may result in signal distortion due to excessive signal strength. During the last bit of the downlink communication 518, the external controller 202-2 may set the VGA gain, at block 528, based on the estimation made from the first downlink bit.

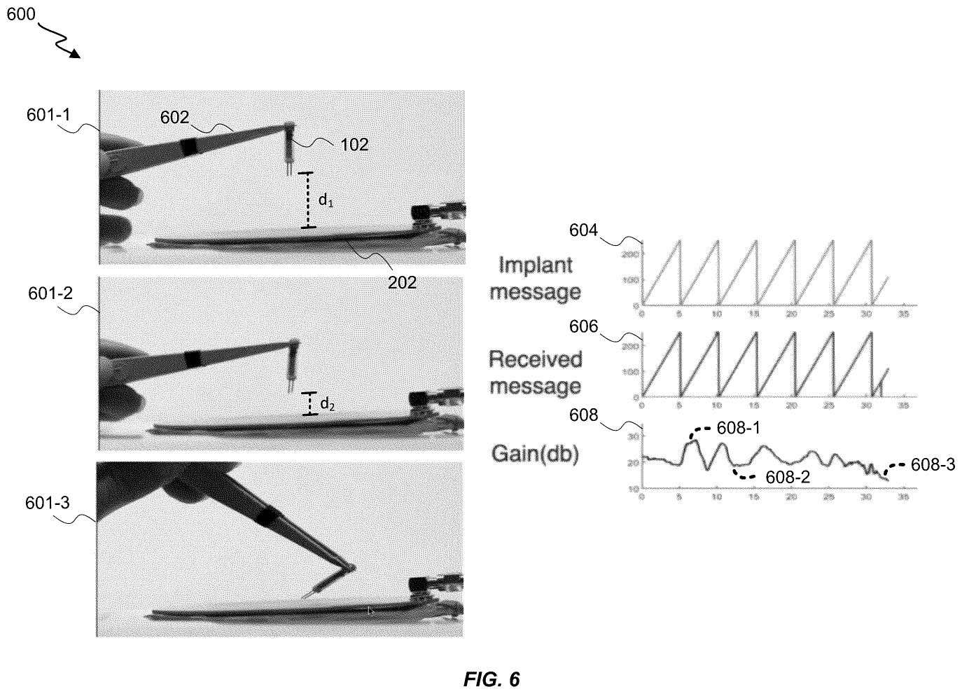

FIG. 6 illustrates screenshots from a practical demonstration of the adaptive voltage control mechanism for the MEWB 102 in accordance with some embodiments of the present disclosure. As discussed before, Adaptive voltage control mechanism 500 can dynamically adjust its amplification gain based on the distance between transmitter coil 108 and the MEWB 102.

FIG. 6 illustrates this dynamic adjustment in gain involving the MEWB 102 held by a tool (e.g., tweezer or tong for movement and positioning of MEWB 102) and an external transceiver 202, comprising transmitter coil 108, receiver coil 212 and the external controller 202-2, placed horizontally at a surface. For the practical demonstration, corresponding waveform of a test message 604, referred as “Implant message” is shown. The received signal by the external transceiver 202 is displayed as “Received message” waveform 606. Due to continuous up and down movements of the hand holding the tool 602, the distance between the external transceiver 202 and MEWB 102 is not constant and is changing continuously. These changes in distances are also reflected in the gain waveform 608 measured in decibels (dB). Three screenshots, 601-1, 601-2, 601-3 were taken at three distances while the hand was moving. The corresponding gains for various distances are shown in decibels (dB) including three notable points: 608-1, 608-2, 608-3. In the first screenshot 601-1, MEWB 102 is held at a larger distance d1 above the external transceiver 202 that corresponds to the point 608-1 depicting larger gain. In the second screenshot 601-2, MEWB 102 is moved closer to the external transceiver 202 and the corresponding gain 608-2 is also reduced. As the MEWB 102 is brought closer, almost touching the external transceiver 202, the corresponding gain 608-3 is also appearing to be approximately zero while avoiding saturation.

### Example Implementation

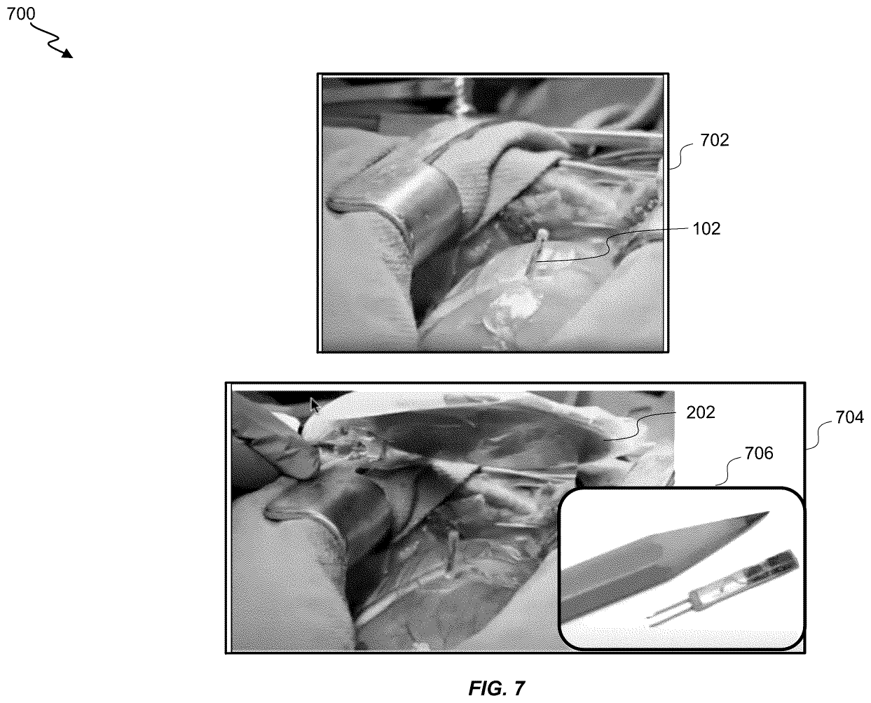

An example design of the MEWB 102 is manufactured and tested in-vivo for a beating heart porcine model to record the electrical activities of heart. FIG. 7 illustrates screenshots of an in-vivo open-heart surgery demonstration 700 showing the MEWB 102 implanted in a living porcine heart to capture electrogram (EGM) activities. MEWB was tested to demonstrate cardiac sensing efficacy and the potential for clinical translation. It may be understood that though the demonstration of MEWB 102 device is illustrated for measuring cardiac activities in the present disclosure, it may also be implanted at other organs of the body 104. As can be seen in screenshot 702 of FIG. 7, the chest is opened surgically, and the device deployment is epicardial (i.e., at the heart surface) using needle electrodes. It is also worth mentioning that the device can be designed to be delivered endovascularly through the femoral vein and anchored within the heart chamber with reduced invasive procedures. However, the open-heart surgery demonstration 700 is to validate the robust operation of the device and evaluate the quality of the recorded signal. In screenshot 704, external transceiver 202 including transmitter coil 108 was then brought to the surface of the skin to power the MEWB 102 and receive the recorded EGM signal wirelessly.

An illustrative image 706 is also shown in FIG. 7 in which MEWB 102 is placed next to a pencil for size comparison, thus highlighting compactness and miniature scale manufacturing of MEWB 102. After wire bonding, MEWB 102 was encapsulated with medical-grade polyurethane epoxy to maintain the structural integrity of the bonds. The sensing electrodes 206 are kept outside the encapsulated area making MEWB 102 to be 2.5 cm long (including electrodes) and 3 mm in diameter. This design is adopted to enable delivering the device endovascularly with a catheter of 12 Fr (4 mm) or less. The characteristic of MEWB 102 is that it is battery-free and externally powered, which allows the device to be much smaller, enabling more devices to be placed within the organ, for example, when implanted for heart, MEWB 102 may be used for multisite pacing or painless defibrillation eliminating replacements due to battery depletion.

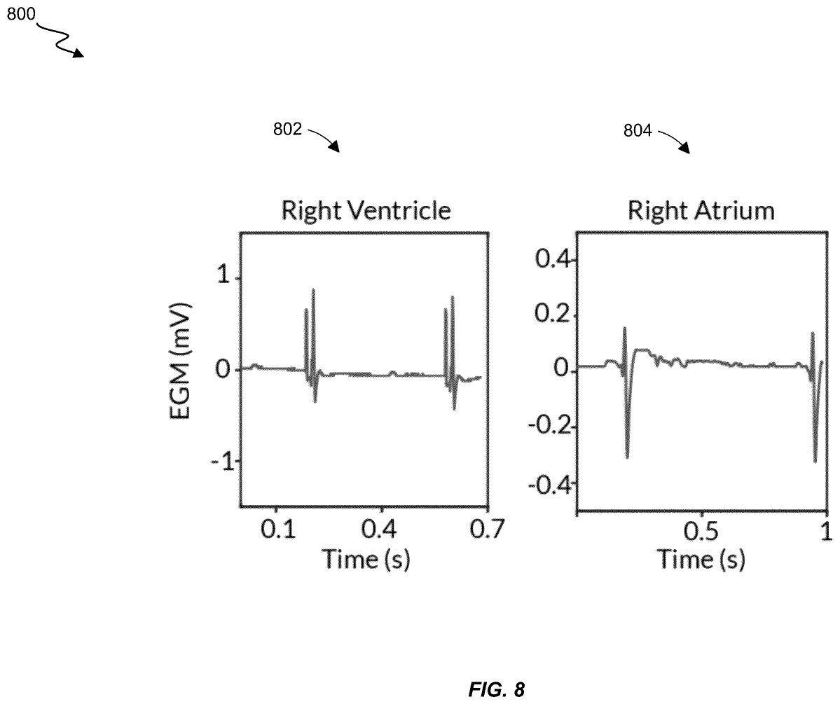

FIG. 8 shows waveforms of captured EGM 800 from the in-vivo demonstration of the FIG. 7. The EGM activity is being recorded while the MEWB 102 device was placed at the heart surface, experiencing dynamic movement associated with the heartbeats. Plot 802 shows two distinct peaks indicating ventricular polarization and repolarization phases in time-domain illustrating EGM activities for the right ventricle plotted along y-axis and measured in mV. To capture the EGM activities in the right atrium, the device was further moved to the right atrium. Plot 804 shows the EGM activities for the right atrium showing atrial depolarization and repolarization phases, typically with smaller amplitudes as compared to ventricular signals due to thinner atrial walls and different conduction characteristics of atrial tissue. The polarity of these recorded signals may be influenced by electrode placement, orientation and the intrinsic nature of atrial cardiac electrical activities. These waveforms of captured EGM 800 confirm the efficacy of the disclosed MEWB device for cardiac activity measurements.

In this exemplary design, the charge storing element 208 is a capacitor mounted on a printed circuit board (PCB) before wire bonding the customized SoC 210 to the board. The magnetoelectric (ME) transducer 204 was fabricated with a 127 μm thick PZT (APC Int.) as a piezoelectric material bonded to a 23 μm thick layer of unannealed Metglas (2605SA1, Metglas) as a magnetostrictive layer 214 with a thin epoxy layer (Hardman Double/Bubble). The two layers were then laser cut by a femtosecond laser cutter to the targeted shape to operate in the 300-400 kHz frequency range, at which the ME transducers 204 operate in the fundamental extensional vibration mode instead of bending mode. The extensional vibration mode corresponds to the vibrations where the material expands or contracts along the length or longitudinal axis while the bending mode refers to the vibrations where the material bends or flexes, typically perpendicular to its surface. Though the bending mode has higher magnetoelectric energy conversion efficiency, longitudinal resonance modes yield slightly higher voltage coefficients under the influence of alternating magnetic field 106.

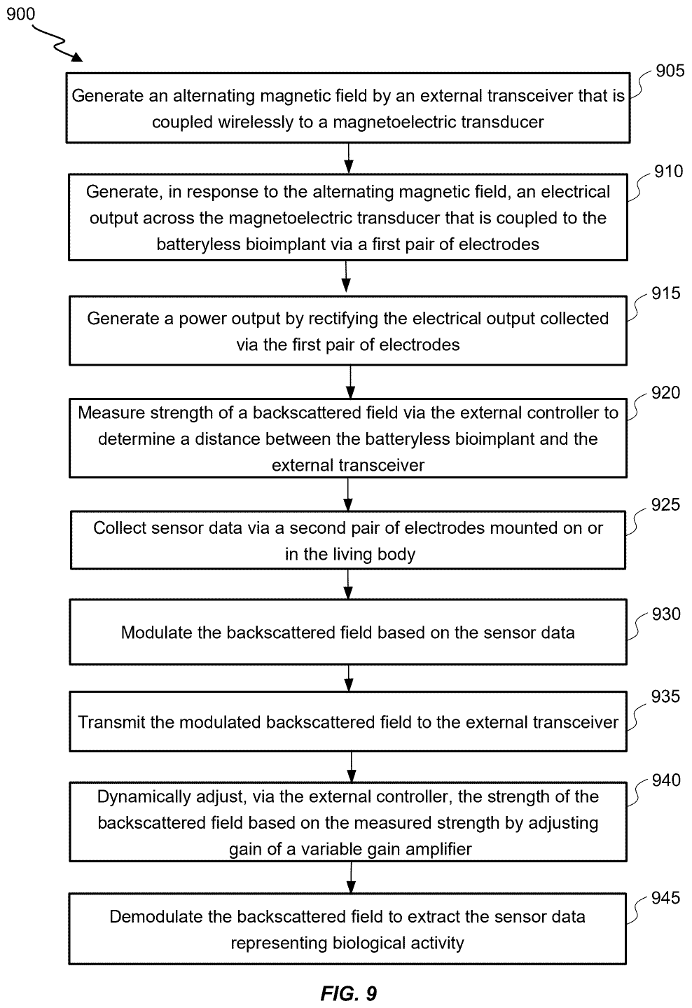

FIG. 9 illustrates an exemplary workflow of the disclosed magnetoelectric wireless bioimplant (MEWB) 102. The blocks in workflow 900 are illustrated in a specific order, while the order can be modified, for example, some blocks may be performed before other, and some blocks may be performed simultaneously. The block can be performed by hardware, software, or a combination thereof. For sensing a biological activity using the disclosed implant, an alternating magnetic field 106 may be generated, at block 905, by an external transceiver 202 that is coupled wirelessly to the ME transducer 204. The external transceiver 202 may comprise a transmitter coil 108, a receiver coil 212 and an external controller configured to control operations of the MEWB 102 remotely and govern working of the external transceiver 202. At block 910, the alternating magnetic field 106 may generate an electrical output (VAC) across the ME transducer 204 due to magnetoelectric coupling, which induces a mechanical strain. The ME transducer 204 may be attached to the MEWB 102 via a first pair of electrodes 205 mounted across the surface of the ME transducer 204. The electrical output (VAC) may be collected via these first pair of electrodes 205 and rectified to generate a power output (V_{rec}), at block 915.

Under the influence of external alternating magnetic field 106, ME transducer 204 may exhibit emission of waves that may reflect back to the source (transceiver) resulting in a backscattered field generated by the ME transducer 204. At block 920, the external controller may measure the strength of this backscattered field during the first bit of downlink communication 518 to determine a distance between the MEWB 102 and the external transceiver 202. The sensor data including the biological activities may be collected via sensing electrodes 206, mounted on or in the living body 104, at block 925. The microcontroller 230 within the MEWB 102 may modulate the backscattered field, at block 930, based on the sensed data by changing the capacitive load of the ME transducer 204 that may be achieved by a switching circuit to electrically connect and disconnect a reactive component based on the sensor data. At block 935, the ME transducer 204 may transmit the modulated backscattered signal to the external transceiver 202 that may receive this signal via the receiver coil 212. The external controller 202-2 may dynamically adjust the strength of the backscattered field based on the measured strength of the backscattered field by adjusting gain of a variable gain amplifier 516, at block 940. To extract the biological activity encoded in the modulated backscattered signal, the external transceiver 202 may demodulate the backscattered signal, at block 945.

Although specific aspects have been described, various modifications, alterations, alternative constructions, and equivalents are possible. Embodiments are not restricted to operation within certain specific data processing environments but are free to operate within a plurality of data processing environments. Additionally, although certain aspects have been described using a particular series of transactions and steps, it should be apparent to those skilled in the art that this is not intended to be limiting. Although some flowcharts describe operations as a sequential process, many of the operations can be performed in parallel or concurrently. In addition, the order of the operations may be rearranged. A process may have additional steps not included in the figure. Various features and aspects of the above-described aspects may be used individually or jointly.

Further, while certain aspects have been described using a particular combination of hardware and software, it should be recognized that other combinations of hardware and software are also possible. Certain aspects may be implemented only in hardware, or only in software, or using combinations thereof. The various processes described herein can be implemented on the same processor or different processors in any combination.

Where devices, systems, components or modules are described as being configured to perform certain operations or functions, such configuration can be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes can communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects can provide those skilled in the art with an enabling description for implementing various aspects. Various changes may be made in the function and arrangement of elements.

The specification and drawings are, accordingly, to be regarded in an illustrative rather than a restrictive sense. It can, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

1-6. (canceled)

7. An apparatus of a bioimplant mounted on or in a living body, the apparatus comprising:
a magnetoelectric transducer coupled to an electric circuit through a first pair of electrodes attached across the magnetoelectric transducer, wherein the electric circuit is configured to:
avail power from the magnetoelectric transducer;
send sensor data collected via a second pair of electrodes to an external transceiver; and
receive data from the external transceiver;
a second pair of electrodes attached to the electric circuit, wherein the second pair of electrodes are configured to mount the bioimplant on or in the living body; and
a bias magnet configured to enhance a mechanical strain in the magnetoelectric transducer.

8. The apparatus of claim 8, further comprising:
a switching circuit configured to modulate the magnetoelectric transducer during an uplink communication such that the switching circuit electrically connects and disconnects a reactive component based on the sensor data.

9. The apparatus of claim 8, wherein the magnetoelectric transducer comprises:
at least one layer of magnetostrictive material configured to be magnetized inducing a mechanical strain when an external magnetic field, is applied;
at least one layer of piezoelectric material on the at least one layer of magnetostrictive material configured to generate an electrical signal in response to the mechanical strain from the at least one layer of magnetostrictive material;
an electrode of the first pair of electrodes attached to an outer surface of the at least one layer of magnetostrictive material; and
an other electrode of the first pair of electrodes attached to an outer surface of the at least one layer of piezoelectric material.

10. The apparatus of claim 8, further comprising:
a low noise amplifier with a fixed gain configured to generate a consistent response for measuring a strength of a backscattered signal generated by the ME transducer.

11. The apparatus of claim 10, wherein the at least one layer of magnetostrictive material comprises Metglas.

12. The apparatus of claim 10, wherein the at least one layer of piezoelectric material comprises Lead Zirconate Titanate (PZT).

13. The apparatus of claim 8, wherein the batteryless bioimplant is encapsulated with medical-grade polyurethane epoxy such that the second pair of electrodes are exposed.

14-20. (canceled)

21. Wherein the bioimplant is a batteryless bioimplant.
